(19) United States
(12) Patent Application Publication
(10) Pub. No.: US 2017/0325687 A1
(43) Pub. Date: Nov. 16, 2017

(54) THREE-DIMENSIONAL THERMAL IMAGING FOR MEDICAL APPLICATIONS

(71) Applicant: BAE Systems Information and Electronic Systems Integration Inc., Nashua, NH (US)

(72) Inventors: John R. FRANZINI, Hollis, NH (US); Mark B. LYLES, Exeter, RI (US); Robert H. MURPHY, Lancaster, MA (US)

(21) Appl. No.: 15/522,803

(22) PCT Filed: Aug. 6, 2015
(86) PCT No.: PCT/US2015/043992

§ 371 Date: Apr. 28, 2017

Related U.S. Application Data
(60) Provisional application No. 62/072,554, filed on Oct. 30, 2014.

(51) Int. Cl. A61B 5/00; A61N 5/06; H04N 13/00; A61B 5/01; G03B 35/08

(52) U.S. Cl. CPC A61B 5/0077; H04N 13/0062; A61B 5/015; A61N 5/06; G03B 35/08

(57) ABSTRACT

An apparatus for three-dimensional thermal imaging in medical applications, said apparatus comprising a power supply, user interface controls, focal plane array (FPA), electronics, and optics. It provides two real-time viewable IR channels for binocular vision with a variable focus distance which can be optimized at any distance from six inches to infinity. The present invention enables 3-D vision in the thermal band for greater awareness of everything within the field of view. Potential medical applications are discussed and presented.

### RELATED APPLICATIONS

This Application Claims rights under 35 USC §119(e) from U.S. Application Ser. No. 62/072,554 filed Oct. 30, 2014, the contents of which are incorporated herein by reference. This application is related to U.S. application Ser. No. 13/948,526 filed Jul. 23, 2013, the contents of which are incorporated by reference.

### STATEMENT OF GOVERNMENT INTEREST

The invention was made with United States Government assistance under Contract No. H94003-04-D-0002/0076 awarded by the Department of the Navy. The United States Government has certain rights in the invention.

### FIELD OF INVENTION

This invention relates to thermal imaging and more particularly to three-dimensional (3-D) infrared (IR) imaging.

### BACKGROUND OF THE INVENTION

As is known in the industry, there are a number of ways to achieve three dimensional (3-D) infrared (IR) imaging. One way uses a 3-D scanner and camera using IR light-emitting diodes (LEDs). It uses an image sensor with pixels sensitive in the visual band to acquire a conventional image and pixels sensitive in the IR band to acquire the depth of what is imaged.

Another way relates a 3-D interface using IR light and IR detectors to interact with spatial-temporal data. The apparatus allows a user to model and analyze three-dimensional surfaces by manipulation of glass beads. An array of LEDs under the beads emits IR light through the beads and a camera captures the data.

Another way uses a 3-D thermal imaging system. The apparatus uses two thermal imaging cameras. It uses a master camera and a subservient camera which corrects gain and offset of the master camera. It combines temperature data with 3-D thermal imaging data to provide a 3-D thermal image.

An improved way, however, is still necessary to achieve high-quality 3-D IR images for use in medical applications. Thus, a heretofore unaddressed need exists in the industry to address the aforementioned deficiencies and inadequacies.

### SUMMARY OF THE INVENTION

The present invention is an apparatus for three-dimensional thermal imaging in medical applications. This apparatus includes an imaging device or FPA sensitive to thermal radiation, a power supply, control switches and/or user interface controls, electronics, an image display, objective optics and display optics. It provides two real-time viewable IR channels for binocular vision with a variable focus distance which can be optimized at any distance from six inches to infinity. The present invention enables 3-D vision in the thermal band for greater awareness of everything within the field of view (FOV) from very close to distant objects and scenes.

The present disclosure can also be viewed as providing a method of presenting anatomical features to medical personnel performing a medical procedure on a patient. In this regard, one embodiment of such a method, among others, can be broadly summarized by the following steps: stereographically imaging the anatomical feature so as to provide two stereo infrared channels of images; and displaying the images carried by the infrared channels on a 3-D monitor, such that the 3-D representation of said anatomical feature is accentuated both as to identity and as to depth by the 3-D representation.

The present disclosure can also be viewed as providing a medical imaging system for providing 3-D representations of thermal images of a subsurface anatomical feature of a patient. Briefly described, in architecture, one embodiment of the system, among others, can be implemented as follows. A 3-D monitor presents a three-dimensional image on a screen thereof. A pair of co-located infrared cameras each has an optical axis, each of said infrared cameras, having an output. A housing for said infrared cameras includes a subassembly for skewing the optical axes of said cameras to impinge on a point spaced from said cameras and adapted to detect one of said anatomical features thereat. A pair of optical image transmission channels is each coupled to a different one of said infrared cameras at one end and said 3-D monitor at the other end for inputting to said 3-D monitor a pair of stereoscopic images such that a stereoscopic image is presented on said 3-D monitor of said anatomical feature to show said anatomical feature and the depth of said anatomical feature in a three-dimensional representation of said anatomical feature.

### BRIEF DESCRIPTION OF THE DRAWINGS

These and other features of the subject invention will be better understood in connection with the Detailed Description in conjunction with the Drawings of which:

FIG. 1 is a perspective drawing showing a preferred embodiment of a dual channel imager system of the invention with an example user setting;

FIG. 2 is a diagrammatic illustration of the two infrared channel system which is utilized to drive a 3-D monitor for the display of subsurface anatomical features of a patient undergoing examination and/or treatment;

FIG. 3 is a diagrammatic illustration of a stereoscopic two channel infrared detection system for use in the system of FIG. 1 showing side-by-side, infrared cameras and focal plane arrays, with each of the cameras being adjustable and focused on to near in objects to provide high quality infrared imaging;

FIG. 4 is an internal view of a dual channel imager showing major components including the adjustment of the two cameras relative to each other to provide near in focusing; and,

FIG. 5 is a block diagram of the dual channel imager shown in FIG. 4, showing the stereoscopic camera and the two optical channel transmission system for coupling the output of the camera to an analog video monitor for the presentation of the three-dimensional image.

### DETAILED DESCRIPTION

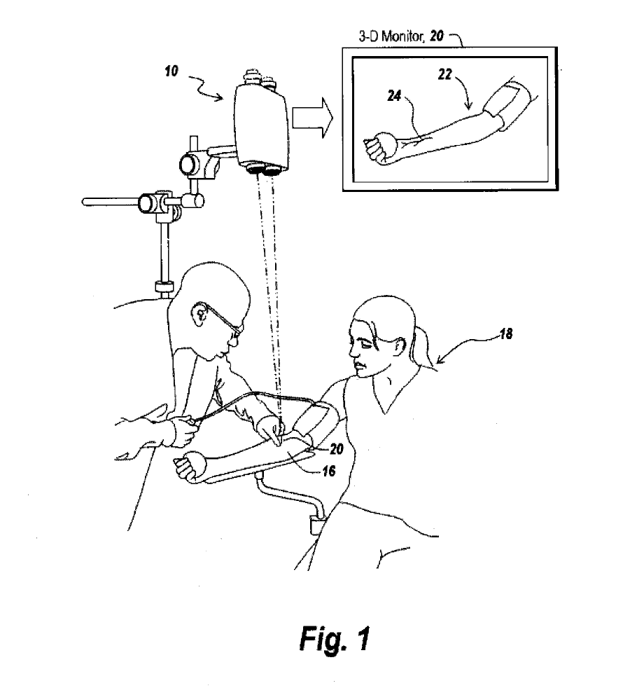

FIG. 1 is a perspective drawing showing a preferred embodiment of a dual channel imager system of the invention with an example user setting. Specifically, FIG. 1 is an example diagrammatic illustration of the utilization of the subject binocular infrared system for identifying blood vessels in the arm of a patient undergoing a phlebotomy. The presented invention is envisioned to have utility in identifying blood vessels in the arm of a patient and other medical procedures where depth perception is important as a diagnostic aid.

In the illustrated system, subsurface anatomical features, for instance, in the arm 16 of a patient 18 are detected through a binocular infrared camera system 10 which is focused on the subsurface region of the patient's arm as illustrated at 16. Here, the output of camera 10, is coupled to a 3-D monitor 20 which produces a three-dimensional image 22 of the patient's arm, and more particularly, a subsurface vein, such as vein 24 which is shown in three dimensions to be a certain distance from the surface of the patient's arm. This representation of subsurface anatomical features is an improvement over the presentation of a two-dimensional image in that by viewing the monitor a physician can obtain a sense of the depth of the anatomical feature. Note that any conventional 3-D monitoring system which has stereoscopic channels as inputs is within the scope of the subject invention. The system is useful not only in the phlebotomy example shown, but also is useful in surgical procedures to give the surgeon a three-dimensional view of the subsurface anatomical feature to be operated on.

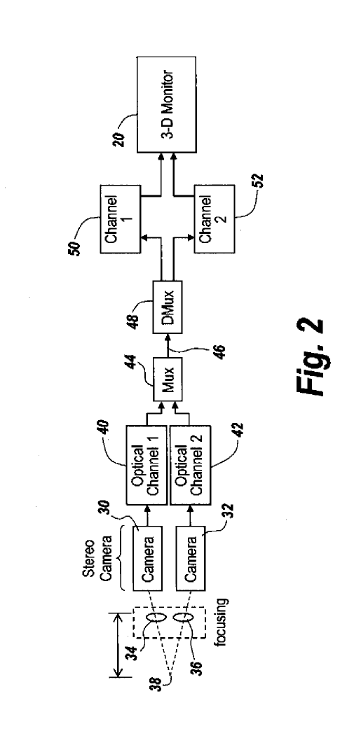

FIG. 2 is a diagrammatic illustration of the two infrared channel system which is utilized to drive a 3-D monitor for the display of subsurface anatomical features of a patient undergoing examination and/or treatment. While the features of the stereo infrared camera are shown in U.S. patent application Ser. No. 13/948,526 as well as its ability to focus in on near in subsurface objects through the canting of the two individual cameras utilized, as shown in FIG. 2, the stereo camera is comprised of cameras 30 and 32 having objective lenses, respectively 34 and 36, that can be focused to a point 38. The output of each of these cameras is applied to a first optical channel 40 and a second optical channel 42 which are multiplexed at 44 and transmitted as illustrated at 46 to a demultiplexing circuit 48. The output of demultiplexing circuit 48 reconstructs optical channels 40 and 42 as optical channels 50 and 52 which are coupled to a conventional 3-D monitor such as monitor 20 of FIG. 1.

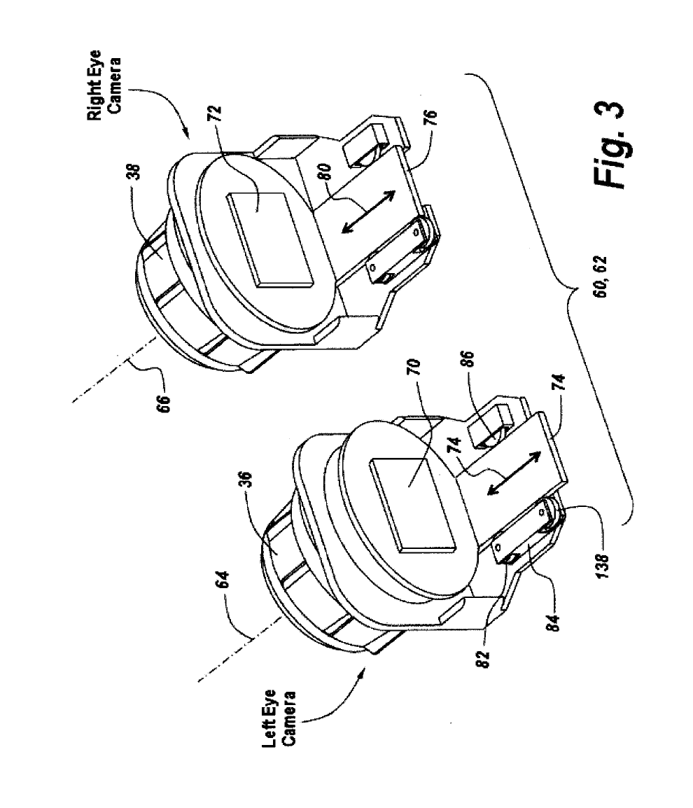

FIG. 3 is a diagrammatic illustration of a stereoscopic two channel infrared detection system for use in the system of FIG. 1 showing side-by-side, infrared cameras and focal plane arrays, with each of the cameras being adjustable and focused on to near in objects to provide high quality infrared imaging. Referring to FIG. 3, in one embodiment, the stereoscopic camera of FIG. 1 includes two separate cameras 60 and 62, each having an optical center line, respectively 64 and 66, which are aimed at point 38 in FIG. 2. Cameras 60 and 62 have individual FPGAs 70 and 72 mounted on respective carriages 74 and 76, with the carriages movable in the direction of double ended arrows 78 and 80 respectively. In this embodiment, the carriages are supported by a wheeled structure 82 having a pair of wheels 84. On the other side of carriage 74 and 76 is a drive wheel structure 86 to provide for focusing in each of the optical channels provided by these two cameras.

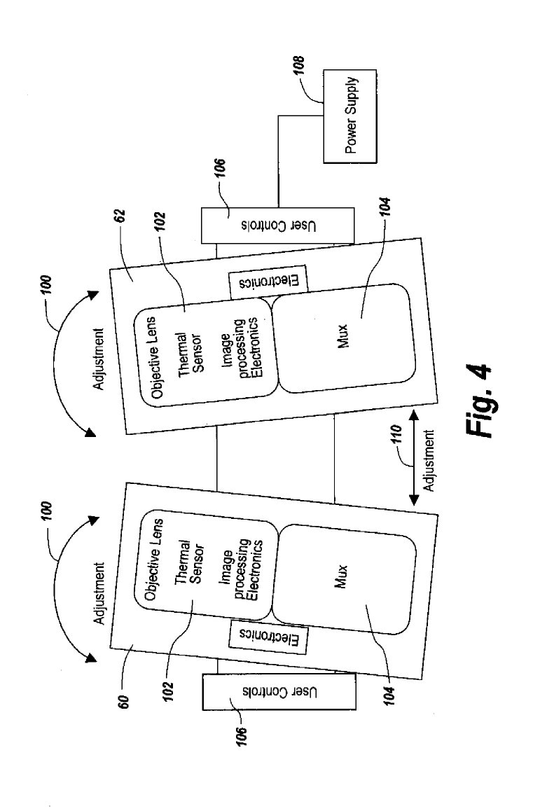

FIG. 4 is an internal view of a dual channel imager showing major components including the adjustment of the two cameras relative to each other to provide near in focusing. From a diagrammatic point of view, and referring now to FIG. 4, each of the cameras 60 and 62 are mounted for securing adjustment, as illustrated by double ended arrows 100 so that the optical center lines of these cameras can be directed to a predetermined point. Each of these cameras includes an objective lens, thermal sensor, image processing, and electronics 102, and MUX circuits 104 adapted to be connected to monitor 20. Coupled to the camera are user controls 106 and a power supply 108, with the adjustment of the pointing direction of each of these cameras being adjustable, as illustrated by double ended arrow 110 so as to be able to cant the cameras with each respect to each other.

The objective optics focuses the thermal scene onto the FPA. The lens focus is adjustable from a near object distance of 6 inches to a far object distance of infinity.

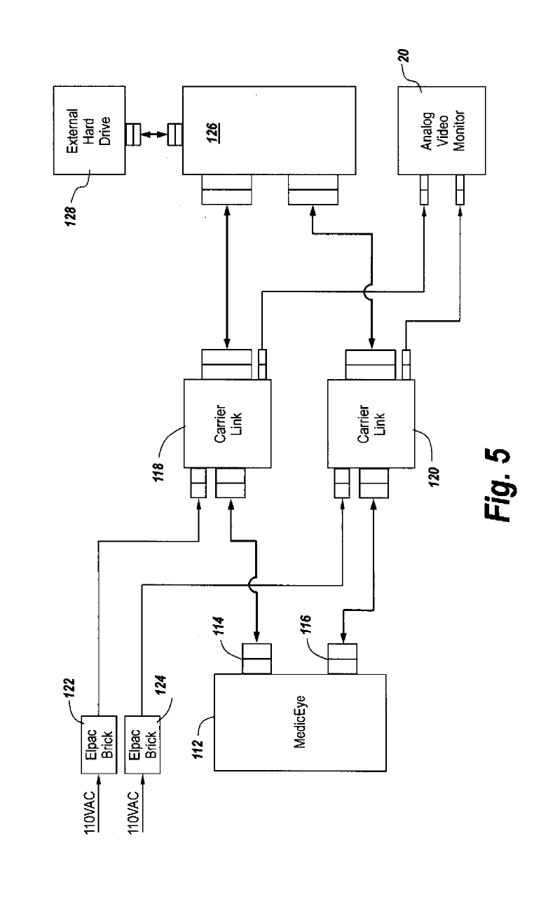

FIG. 5 is a block diagram of the dual channel imager shown in FIG. 4, showing the stereoscopic camera and the two optical channel transmission system for coupling the output of the camera to an analog video monitor for the presentation of the three-dimensional image. Referring now to FIG. 5, in one embodiment, the stereoscopic infrared camera shown is MedicEye 112 having two output ports 114 and 116 coupled to respective camera link modules 118 and 120 to which power is supplied by respective Elpack bricks 122 and 124. Camera link modules 118 and 120 are coupled to a computer 126 having an external hard drive 128, with the output of the camera link modules being applied to an analog video monitor 20 for the purpose of presenting the required 3-D image to the medical professional.

In operation, and referring now to the system level block diagram of FIG. 5, this diagram shows the major electrical interfaces and how the communication protocol is implemented. Long wave infrared (LWIR) data is transmitted from the LWIR cameras of MedicEye camera 112 to the MedicEye computer 126 over a CameraLink interface involving camera link modules 118 and 120. An Imprex dual PC-Express frame grabber installed in the MedicEye computer and associated FrameLink Software (SW) enables real-time monochrome display of data and data stream capture in the two optical channels in the form of a numbered TIF sequence. Gain & level of the displayed data is controlled by adjusting the FrameLink high & low histogram points. Note that the FrameLink software is described in PCT/US2014/060897 filed Oct. 16, 2014 entitled Medical Thermal Imaging Processing for Vein Detection incorporated herein by reference.

It will be appreciated that by providing stereoscopic information to a 3-D monitor, the result is a three-dimensional image portrayed on the monitor which is useful for the medical community to be able to visualize the position of subsurface features and to be able to conduct either diagnosis or treatment, including surgery, in a manner in which two-dimensional displays are incapable.

The subject system may be utilized for intravenous vessel detection, bone ablation and deburring, bleed detection during surgery and dental health procedures, including detection of tooth health by direct IR imagery. This may also include the use of reflective technology, including, an IR dental mirror.

While the present invention has been described in connection with the preferred embodiments of the various Figures, it is to be understood that other similar embodiments may be used or modifications or additions may be made to the described embodiment for performing the same function of the present invention without deviating therefrom. Therefore, the present invention should not be limited to any single embodiment, but rather construed in breadth and scope in accordance with the recitation of the appended Claims.

1. A medical imaging system for providing 3-D representations of thermal images of a subsurface anatomical feature of a patient, comprising:
a 3-D monitor for presenting a three-dimensional image on a screen thereof;
a pair of co-located infrared cameras, each having an optical axis, each of said infrared cameras, having an output;
a housing for said infrared cameras, including a subassembly for skewing the optical axes of said cameras to impinge on a point spaced from said cameras and adapted to detect one of said anatomical features; and,
a pair of optical image transmission channels, each coupled to a different one of said infrared cameras at one end and said 3-D monitor at the other end for inputting to said 3-D monitor a pair of stereoscopic images such that a stereoscopic image is presented on said 3-D monitor of said anatomical feature to show said anatomical feature and the depth of said anatomical feature in a three-dimensional representation of said anatomical feature.

2. The system of claim 1, wherein each of said cameras includes a focusing module focusing each of said cameras on said point.

3. The system of claim 1, wherein each of said cameras includes a high resolution infrared camera having a high resolution focal plane array.

4. The system of claim 1, wherein each of said cameras has a focal plane array and an objective lens, and wherein said focusing module includes a carriage for the associated focal plane array and a movement device for translating said carriage to move said focal plane array with respect to the associated objective lens to effectuate focusing.

5. The system of claim 1, wherein said pair of optical transmission channels includes a multiplexing circuit for multiplexing said two optical channels and a demultiplexing circuit for demultiplexing the multiplexed optical channels prior to coupling to said monitor.

6. The system of claim 1, wherein said point is between 6 inches and an infinite distance from said cameras.

7. The system of claim 1, wherein said system detects intravenous vessels to facilitate blood draw.

8. The system of claim 1, wherein said system cools bone and related tissue during ablation and deburring methods.

9. The system of claim 1, wherein said system detects bleeding during surgery, wherein the bleeding is not evident with a human eye.

10. The system of claim 1, wherein said system, during dental procedures, detects dental health and vitality.

11. The system of claim 10, wherein the imaging apparatus detects dental health by at least one of: direct IR imaging, IR reflective techniques, and IR dental mirror.

12. A method of presenting anatomical features of a medical procedure on a patient, comprising:
stereographically imaging the anatomical feature so as to provide two stereo infrared channels of images; and,
displaying the images carried by the infrared channels on a 3-D monitor, such that a 3-D representation of said anatomical feature is accentuated both as to identity and as to depth by the 3-D representation.

13. The method of claim 12, wherein the infrared channels are generated through the use of a stereo infrared camera having two infrared optical cameras having optical center lines focused on the anatomical feature.

14. The method of claim 13, wherein the two infrared cameras are skewed such that the optical center lines thereof converge on a single point.

15. The method of claim 13, wherein each of the two infrared cameras have separate focusing adjustment mechanisms.

16. The method of claim 12, further comprising multiplexing the two stereo infrared channels of images.

17. The method of claim 12, further comprising displaying the 3-D representation of the medical procedure to medical personnel.
